SEQUENCE LISTING (3 sequences)

SEQ ID NO: 1 - DNA, 1422 bp, Artificial Sequence
  Description of Artificial Sequence Synthetic polynucleotide
gccatggactggacctggagcatccttttcttggtggcagcagcaacaggtgcccactcccaggttcaactggtgcagtctggagctgaggtgaagaagcctggggcctcagtgaaggtctcctgcaaggcctctggttacacctttagcagcaacggtatcatctgggtgcgacaggcccctggacaagggcttgagtggctgggatggatcagcggttacaatggtaaaacaaggtatgcacagaaggtccagggcagagtcaccattaccacagacacatccacgagcacggcctacatggagctgaggagcctgagatctgacgacacggccgtgtattactgtgcgagagaaaaaactatggttcggggagctatatctgggtatagtgactactggggccagggaaccctggtcaccgtctcctcagcaagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 2 - protein, 474 aa, Artificial Sequence
  Description of Artificial Sequence Synthetic polypeptide
AMDWTWSILFLVAAATGAHSQVQLVQSGAEVKKPGASVKVSCKASGYTFSSNGIIWVRQAPGQGLEWLGWISGYNGKTRYAQKVQGRVTITTDTSTSTAYMELRSLRSDDTAVYYCAREKTMVRGAISGYSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 474 aa, Homo sapiens
AMDWTWSILFLVAAATGAHSQVQLVQSGAEVKKPGASVKVSCKASGYTFSSNGIIWVRQAPGQGLEWLGWISGYNGKTRYAQKVQGRVTITTDTSTSTAYMELRSLRSDDTAVYYCAREKTMVRGAISGYSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0002945 A1
(43) Pub. Date: Jan. 6, 2011

(54) Use of immunoglobulin heavy and light chains or fragments thereof to bind to aggregated amyloidogenic proteins

(76) Inventors: Brian O'Nuallain, Dublin (IE); Scott Dessain, Wynnewood, PA (US); Sharad Adekar, Secane, PA (US)

Correspondence Address: HOWARD EISENBERG, ESQ., 1220 LIMBERLOST LANE, GLADWYNE, PA, 19035, US

(21) Appl. No.: 12/803,384

(22) Filed: Jun. 25, 2010

Related U.S. Application Data
(60) Provisional application No. 61/269,958, filed on Jul. 1, 2009.

(51) Int. Cl. A61K 39/395; C07K 16/18; A61P 25/28

(52) U.S. Cl. 424/172.1; 530/389.1

(57) ABSTRACT

Subunits of antibodies, such as a light chain or a heavy chain, selectively bind to amyloid fibrils and oligomers.

This application claims priority from pending U.S. Provisional Patent Application No. 61/269,958, filed Jul. 1, 2009, which is incorporated herein by reference in its entirety.

### FIELD OF THE INVENTION

The invention pertains to the field of protein misfolding diseases known as amyloidoses, and specifically to the field of antibody binding to fibrils and oligomers present in amyloid.

### BACKGROUND OF THE INVENTION

Amyloidoses are a group of pathologic processes in which normally soluble proteins of diverse chemical composition aggregate in the form of fibrils and are deposited in the brain, heart, liver, pancreas, kidneys, nerves, and other vital tissues, leading to organ failure and, eventually, death. These disorders represent a significant public health problem, most notably in the case of the brain amyloidoses in Alzheimer's Disease (AD). Besides AD, adult-onset (type 2) diabetes, certain forms of cancer (multiple myeloma and the related plasma cell disorder, primary [AL] amyloidosis) and inherited disorders such as familial amyloidotic polyneuropathy, chronic inflammation such as is associated with rheumatoid arthritis and tuberculosis, and the transmissible spongiform prion-associated encephalopathies are representatives of this group of diseases. Additionally, amyloid deposition is a feature of normal aging, such as in senile systemic amyloidosis and cataracts of the eye (Benson et al., 2001; Ross et al., 2004; Enqvist et al., 2003; Meehan et al., 2004).

To date, many different amyloidogenic proteins have been identified (Table 1), for example, immunoglobulin light chains, serum amyloid A protein, β2-microglobulin, transthyretin, cystatin C variant, gelsolin, procalcitonin, PrP (prion precursor) protein, amyloid β-protein, microtubule-associated protein tau, ApoA1, and lysozyme.

TABLE 1
Amyloid Nomenclature: Amyloid fibril proteins
and their precursors in humans

| Amyloid | Protein | Syndrome or Involved Tissue |
|---|---|---|
| Protein | Precursor | (Systemic [S] or Localized [L] |
| AL | Immunoglobulin | Primary (S, L), Myeloma-associated |
| | light chain | |
| AH | Immunoglobulin | Primary (S, L), Myeloma-associated |
| | heavy chain | |
| ATTR | Transthyretin | Familial (S), Senile systemic, |
| | | Tenosynovium (L?) |
| Aβ₂M | β₂-microblobulin | Hemodialysis (S), Joints (L?) |
| AA | (Apo)serum AA | Secondary, reactive (S) |
| AapoAI | Apolipoprotein AI | Familial (S), Aortic (L) |
| AApo AII | Apolipoprotein AII | Familial (S) |
| Agel | Gelsolin | Familial (S) |
| Alys | Lyosozyme | Familial (S) |
| Afib | Fibrinogen α-chain | Familial (S) |
| Acys | Cystatin C | Familial (S) |
| Abri | ABriPP | Familial dementia, British (L, S?) |
| Adan | ADanPP | Familial dementia, Danish (L) |
| Aβ | Aβ protein precursor | Alzheimer's disease, aging (L) |
| AprP | Prion protein | Spongiform encephalopathies (L) |
| Atau | Tau protein | Tauopathies, AD, aging (L) |
| ACal | (Pro)calcitonin | C-cell thyroid tumors (L) |
| AIAPP | Islet amyloid | Islets of Langerhans (L), |
| | polypeptide | Insulinomas |
| AANF | Atrial natriuetic factor | Cardiac atria (L) |
| APro | Prolactin | Aging pituitary (L), Prolactinomas |
| Alns | Insulin | latrogenic (L) |
| Amed | Lactadherin | Senile aortic, media (L) |
| AKer | Kerato-epithelin | Cornea; Familial (L) |
| A(Pin) | Unknown | Pindborg tumors (L) |
| ALac | Lactoferrin | Cornea; Familial (L) |

Although these proteins are unrelated by sequence, the fibrils that they form have several common characteristic properties, including the following: 1) they possess a β-pleated sheet secondary structure; 2) they are insoluble aggregates; 3) they are stained by certain intercalating dyes, e.g. Congo red; and 4) they possess a characteristic unbranching fibrillar structure when observed under an electron microscope.

Polyclonal and monoclonal antibodies (mAb) have been generated that specifically recognize native amyloid precursor proteins. These antibodies bind to particular epitopes of amyloid determined by the amino acid sequence of the precursor proteins. Such sequence specific antibodies may also recognize fibrils of a particular type of amyloid, but do not recognize fibrils from sequence-unrelated types of amyloid.

Polyclonal and monoclonal antibodies (mAb) have been generated that specifically recognize antigenic determinants expressed on amyloid fibrils or soluble oligomeric assembly intermediates, but not the native precursor proteins [e.g. Lambert et al., J. Neurochem. 100, 23-35 (2007); Kayed et al., Mol. Neurodegener. 2, 18 (2007)]. Additionally, IgG or IgM mAbs prepared against light chain (LC) or amyloid β peptide (Aβ)fibrils, or IgGs present in normal, presumably healthy individuals, have been found to cross-react with those formed from unrelated amyloidogenic precursors, such as β₂-microglobulin (β₂M), serum amyloid A protein (SAA), islet amyloid polypeptide (IAPP), transthyretin (TTR), and polyglutamine (polyGln) [Hrncic et al., Am. J. Pathol. 157, 1239-1246 (2000); O'Nuallain and Wetzel, Proc. Natl. Acad. Sci. USA 99, 1485-1490 (2002); O'Nuallain et al., J. Immunol. 176, 7071-7078 (2006); O'Nuallain et al., Biochemistry 47, 12254-12256 (2008)], suggesting that amyloids share generic conformational epitopes unrelated to amino acid sequence.

One of the most relevant diseases exhibiting extensive formation of amyloid depositions are neurodegenerative diseases, like AD (amyloidoses of Aβ peptide and tau protein), Parkinson's disease and Lewy body variant of AD (synuclein aggregation), and prion diseases. Classical AD is distinguished by the fact that two unrelated types of amyloid formation are concomitantly observed: Mostly extracellular deposition of Aβ-peptide in the form of senile plaques (SP), and intracellular accumulation of aggregates of the microtubule-associated protein tau in a biochemically modified form as neurofibrillary tangles (NFT). Aβ peptides are derived from the integral membrane precursor protein APP of poorly understood normal function, while NFT pathology involves the assembly of the neuron-specific microtubule-associated proteins tau into Paired Helical Filaments (PHF). The aggregating domain of tau consists of the microtubule-binding repeats (PHF core domain) in a β-pleated sheet confirmation, which renders PHF an amyloid structure. PHFs are exlusively composed of tau in an abnormal form involving a process of unphysiological hyperphosphorylation. The normal function of tau is believed to be related to neuron-specific adaptions in organizing the microtubule-cytoskeleton to accommodate specific challenges in transport along microtubules (MT) in the extremely extended processes of neurons. PHF-type pathological hyperphosphorylation is known to abolish binding to MT, change tau conformation, and may render tau available for aggregation. Several tau proteins are formed by splicing in a species-specific manner, but only during a process of “adult maturation”. In fetal mammalian brains, only one splice isoform is found. There is a disease specific pattern of involvement of adult splice isoforms in different tau-related neurodegenerations for unknown reasons.

The two amyloid lesions can also occur separately: Aβ amyloidosis is often found in brains of advanced age, and if confined to the brain parenchyma, is usually not associated with severe clinical dementia and obvious neurodegeneration. Dementia is observed if Aβ amyloidosis affects the cerebral vasculature as well (Vascular Dementia). NFT pathology alone, essentially indistinguishable from that of AD, occurs in some 20 rare neurodegenerative diseases, whereby different diseases have distinct regional distributions of pathology in brain, with symptoms varying according to the function of the brain regions affected (certain forms of Frontotemporal Dementia, Progressive Supranuclear Palsy, Pick's Disease, Argyrophilic Grains Disease, Hippocampal Sclerosis and others). In some instances NFT only diseases can be caused by mutations in tau protein, demonstrating that tau can be a primary cause of tau pathology, and that tau pathology alone is sufficient to drive neurodegeneration to an extent and within a time frame similar to AD.

The cause-effect relationships in AD are more complex: APP mutations alone are sufficient, but not necessary, to precipitate both Aβ and NFT pathology, and neurodegeneration, later in life in humans. However, APP mutations in transgenic mice produce only Aβ pathology and no tau pathology, and no overt neurodegeneration. Aβ pathology in mice is associated with focal morphological abnormalities of neurites, and interference with synaptic function, e.g. in the process of Long Term Potentiation (LTP), which appears to be acutely reversible, however. Expression of mutant human tau proteins in transgenic mice, but not of wild-type human tau, can lead to PHF-tau pathology with all the obvious hallmarks of human NFT pathology, like hyperphosphorylation. In good correlation with PHF pathology progressive neurodegeneration is in fact observed in such mice, as in humans. This establishes the fact that tau pathology is self-propagating without support from other pathologies.

Equally complex is the exact mechanism of neurotoxicity, further complicated by the fact that the transgenic mouse data suggest that there may be more than one type of toxicity, a view which can be well reconciled with longstanding observations in human AD. Aβ pathology appears to be associated with steady-state disturbances of neuritic/synaptic function that neurons can recover from, and which may be exaggerated in mouse models where Aβ peptides are overexpressed relative to human pathology. Intracellular PHF-tau, on the other hand, affects the structural integrity of neurons in a more profound way and leads to unrecoverable loss of dendritic arbor and eventually the whole neuron. In aggregate, this is the basis for the macroscopically observed brain atrophy in AD, and is presumably the basis for the irreversible progressive nature of AD and similar diseases.

In either case the precise nature of offending molecular species, and their respective mechanism of toxicity, are also not well understood. It is, however, an increasingly common view that the pathological hallmarks of amyloid plaques and NFT, as endstages of molecular pathologies, are probably less toxic, and may rather represent a successful detoxification of much more problematic precursors. In the case of Aβ peptides oligomeric assemblies are suspected to form molecular pores in membranes and interfere with certain neurotransmitter receptors. For tau, the fact that aggregation is never observed in the absence of hyperphosphorylation, and that PHF never contain co-aggregated normally phosphorylated tau invites the view that hyperphosphorylation is part of rendering the molecule pathogenic. In contrast to Aβ pathology, the mechanism(s) of toxicity are more obscure for abnormal tau species.

Regardless of the precise molecular details of the respective pathologies, the obvious common denominator is protein misfolding at some point in the pathological pathway. In recognition of this fact agents directed against the misfolded proteins are the basis for prospective therapeutic interventions aimed at the causative stages of the respective diseases (foldopathies). In particular antibodies are of use to target conformational abnormalities.

The concept of immunotherapy for AD was introduced with the demonstration that inoculation of transgenic mice overexpressing APP mutants that cause AD in humans with human Aβ peptide leads to the generation of antibodies, which clear amyloid plaques and appear to preserve cognitive function [D. Schenk et al., Nature 400, 173-177 (1999)], but not in aged dogs with native levels of Aβ and plaque deposition [E. Head et al., J. Neurosci. 28, 3555-3566 (2008)]. In a clinical trial, however, this approach led to incidences of brain inflammation in some patients. Although the immunization schedule was stopped, some of patients, which did not suffer from these complications, maintained high anti-Aβ antibody titers and their clinical progression was monitored. There was no evidence of reduced neurodegeneration, however, or evidence of delay of the endstage of AD, in spite of the fact that in post-mortem brains of endstage patients substantial clearance of Aβ plaque pathology was verified [C. Holmes et al., Lancet 372, 216-223 (2008)]. In contrast, NFT were unaffected, suggesting that immunological activity directed exclusively to Aβ is insufficient to impact progression.

Passive immunization with exogenously prepared monoclonal Aβ antibodies also leads to plaque clearance in transgenic mouse models. In these models resolution of Aβ plaques occurs rapidly and cognitive impairment is lifted in a correlated fashion. Yet, no such short-term effects have been observed in ongoing clinical trials.

These studies, albeit lacking in desired efficacy to date, have nonetheless led to an important revision of the longstanding notion that the brain is immune-privileged, and antibodies would not cross the bloodbrain barrier. Moreover, in an analogous extension of the Aβ immunization concept, tau immunization has been applied to transgenic mouse models. Surprisingly, antibodies generated by this vaccination were shown to have access to the intracellular environment [Asuni et al., J. Neurosci. 27, 9115-9129 (2007)], possibly due to damage of affected neurons, suggesting that abnormal tau proteins could in principle be targeted with antibodies.

In view of the aforegoing, there is an urgent need for new antibody therapeutics directed against abnormal folding of proteins rather than any specific antigenic sequence determinant. In the case of AD, this would allow to address both the Aβ as well as the tau related amyloidosis concomitantly for greater therapeutic benefit.

Antibodies are gamma globulin proteins and are made of several structural units. The basic unit of an intact antibody is a “Y” shaped structure that contains four polypeptide chains, two identical heavy chains (HC) and two identical light chains (LC) (FIG. 1). HC have four subdomains C_{H}1 to C_{H}3, and the variable domain V_{H} which participates in determining the sequence-directed epitope specificity of the antibody. LC consist only of one C_{L} and one variable V_{L} domain. Each HC pairs with one complementary LC and is linked by a disulfide bridge at the end of the C_{H}1 and C_{L} domains. Two such HC/LC assemblies homodimerize by two neighboring disulfide bridges in C_{H}2 near the C_{H}1 junction. In this functional antibody structure two regions are distinguished, connected by a hinge: the F_{ab} (fragment, antibody binding) region and the F_{c} (fragment, crystallizable) region consisting of the C_{H}2 and C_{H}3 subdomains, which is shared among subclasses of antibodies (IgG1, IgG2 etc.). This constant region is not believed to be involved in specific binding of an antibody to an epitope, but is known to engage certain immune cells into a response to a bound antigen, such as clearance, phagocytosis, complement activation, inflammatory response, etc.

### SUMMARY OF THE INVENTION

It has been unexpectedly discovered that it is a generic property of immunoglobulin (Ig) γ heavy chains, unconnected to an antibody light chain like normal antibodies, have a useful binding activity to a variety of amyloid-forming proteins and peptides independent of primary structure. Such antibody heavy chains, regardless of the intact antibody from which they are derived, are capable of specifically binding to amyloid fibrils and oligomers from any and all amyloidogenic proteins, a property previously not appreciated

Equally unexpected is the discovery that an Ig light chain, unconnected to an antibody γ heavy chain, also have a generic binding activity to amyloid fibrils and oligomers from any and all amyloidogenic proteins, regardless of the intact antibody from which they are derived.

It has been further unexpectedly discovered that the capability of specific binding to amyloid fibrils and oligomers is not dependent upon the source of the Ig heavy chain or light chain. Although anti-amyloidogenic potency may vary between different γ heavy chains or between different light chains, any isolated Ig heavy chain or light chain may be used in principle to bind to amyloid fibrils or oligomers from any and all amyloidogenic proteins.

Thus, in one embodiment, the invention is a method for selectively binding an aggregated amyloidogenic protein, such as an amyloid fibril or oligomer. According to this method of the invention, the aggregated amyloidogenic protein is exposed to an antibody heavy chain or an antibody light chain and the heavy or light chain is permitted to bind to the amyloid. The exposure of the amyloidogenic protein to the antibody heavy chain or light chain may be in vivo or may be in vitro. The exposure may be for diagnostic or therapeutic purposes. Thus, as with conventional intact antibodies, the antibody heavy or light chain may be tagged or coupled with a diagnostic marker or with a therapeutic agent.

In another embodiment, the invention is a method for reducing the toxic effects of a mis-folded peptide in an amyloid related disease. According to this embodiment of the invention, antibody heavy chains or antibody light chains are administered to a subject suffering from an amyloid related disease in an amount sufficient to reduce the effects of the amyloid related disease in the individual. Preferably, the subject is a human. Other animals are also suitable for this embodiment of the invention, including domestic animals, such as dogs and cats, and laboratory animals, such as rodents like mice, rats, and guinea pigs, rabbits, and non-human primates such as monkeys.

In other embodiments, the invention is an antibody heavy chain in its monomeric form, its dimeric form, or any mixture thereof.

In yet another embodiment, the invention is an active fragment of an IgG heavy chain, comprising the either the C_{H}1, C_{H}2, C_{H}3, or V_{H} domain, or any combination thereof, or the fragment C_{L} or V_{L} of an antibody light chain, as depicted in FIG. 1. Preferred combinations are fragments consisting of the V_{H} and C_{H}1 domain, lacking the effector domains of the F_{C} region.

In a preferred embodiment the antibody heavy chain is a specific Ig γ1 chain referred to as F1, with the cDNA and primary protein structure disclosed in FIG. 2. Another preferred embodiment is a fragment consisting of the V_{H} and C_{H}1 domain of the heavy chain F1.

In another preferred embodiment the antibody heavy chain is F1 with any one, two or three of the Cys227, Cys233 and Cys236 residues, which stabilize the interaction with light chains and the homo/hetero-dimerization of heavy chains, mutated to Ala or Ser such as to prevent inactivation of F1 by interchanging with endogenous serum IgGs.

### BRIEF DESCRIPTION OF THE DRAWINGS

FIG. 1 is a schematic representation of an intact antibody structure and subdomain organization.

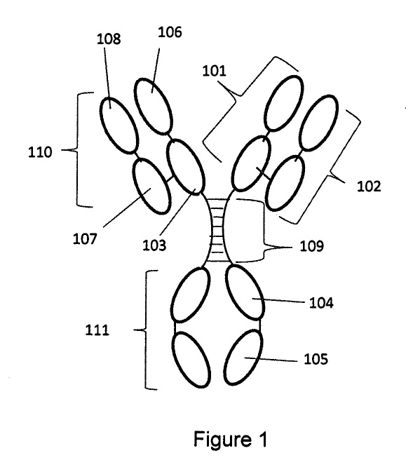

FIG. 2 shows the alignment of the cDNA and protein sequence of antibody heavy chain F1. The underlined amino acid residues are mutated relative to the generic IgG1 heavy chain sequence. The gray-shaded sequence is the V_{H} variable domain.

FIG. 3 is a series of graphs showing (A) Binding of F1 heavy chain Ab to plate-immobilized fibrils of λ6 J to LC (◯), Aβ1-40 (), CAPS (ΔΔ), and non-amyloid elastin aggregates (□). (B) F1 non-specific binding to plate-immobilized Aβ monomer in the presence (◯) or absence () of 100-fold excess soluble Aβ. (C) Aβ1-40 fibril binding by the intact mAb 13A (), heavy chain HC 13A (◯), and F1 HC (▴). (D) Aβ1-40 fibril binding by the intact mAb 30B (), heavy chain HC 30B (◯), and F1 HC (▴). (E) Aβ1-40 fibril binding by polyclonal heavy chains from three human subjects (◯,,□), vs. binding by intact polyclonal IgGs from two subjects (⋄,♦).

FIG. 4 is two graphs showing the binding to plate-immobilized Aβ fibrils of the isolated light chains LC (▪) of (A) mAb 13A and (B) mAb 30B, and of the respective intact antibodies ().

FIG. 5 is a series of graphs showing (A) Dose-dependent inhibitory effect on Aβ1-40 fibril elongation of F1 HC (▴), intact mAb 13A (♦), isolated HC 13A (◯), and BSA control (⋄). (B) LTP after high frequency conditioning stimulation in vivo in the rat hippocampus injected with vehicle () and 40 pmole Aβ1-42 (▴). (C) Prevention of LTP inhibition by Aβ1-42 after co-injection of 60 pmole F1 HC (Δ), but not after co-injection of a similar amount of intact mAb 13A (◯).

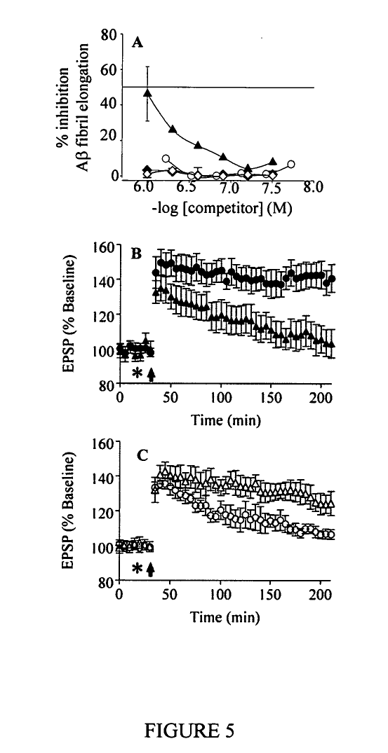

### DETAILED DESCRIPTION OF THE INVENTION

All immunological approaches for AD to date are based on sequence-directed antibodies against Aβ peptides. As current experience in the art shows, there are problems with efficacy in human patients, perhaps related to the fact that such agents may bind to both the aggregated as well as unaggregated antigen without necessarily interfering with what is now believed to be the true toxic event, i.e. the abnormal folding into a β-pleated sheet conformation. This lack of discrimination may also lie at the heart of unintended and unpredictable side effects, presumably arising from binding to the normal antigen, performing its physiological function, or directing an immune attack against the site exhibiting the normal antigen. Efficacy may also be elusive due to the fact that the progress of neurodegeneration, the key feature distinguishing AD from other reversible memory impairments, is more directly tied to the pathology of tau protein rather than Aβ.

Recently a preparation of total immunoglobulin from pooled human blood (IVIG) has shown anti-neurodegenerative activity in small human trials, not seen with any of the Aβ-directed immunological approaches to date [N. R. Relkin et al., Neurobiol. Aging 30, 1728-1736 (2008)]. Analysis of such immunoglobulin preparations has revealed that they contain a minor proportion (0.1-0.2%) of antibodies, which have the ability to bind to a variety of amyloid-forming mis-folded proteins with β-pleated sheet conformation independent of sequence, apparently directed against epitopes common to all β-pleated sheet structures [B. O'Nuallain et al., J. Immunol. 176, 7071-7078 (2006)]. The low abundance of these antibodies may explain the need for exorbitant doses of the IVIG preparation for a therapeutic effect (about 0.4 g/kg per bi-monthly infusion).

The use of immunoglobulin preparations from blood donors for the treatment of AD is subject to severe limitations. The amount of immunoglobulin to be infused on a regular schedule is extremely high, presenting problems related to viscosity of the blood. Donor blood may be contaminated with infectious agents. The amount of donor blood as a source of immunoglobulin preparations is by far too limited and expensive for wide spread use in a mass indication like AD. Consequently there is an urgent need to identify the precise molecular nature of the active principle in such preparations, and make it available in recombinant form, allowing for controlled and reproducible dosing of a pure and scalable therapeutic product.

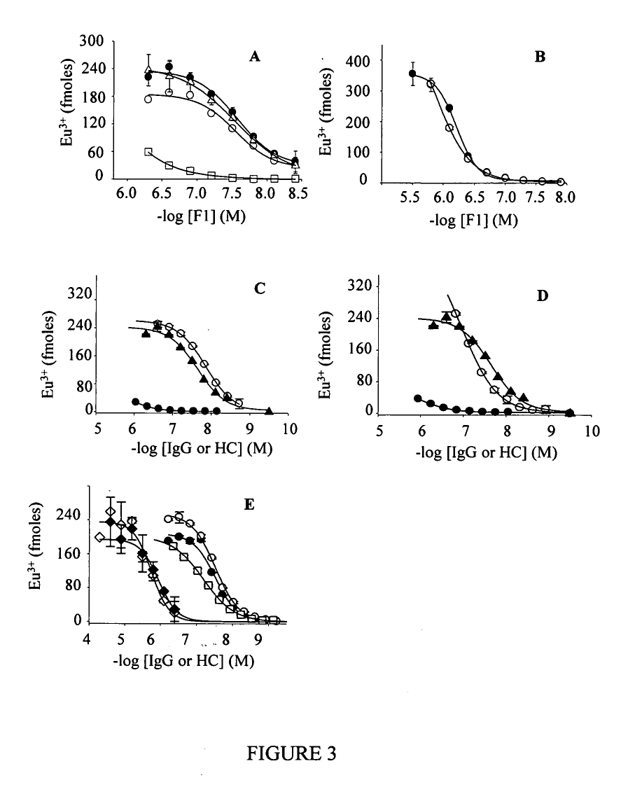

In an effort to identify the active agent in human immunoglobulin fractions with general β-pleated sheet binding activity splenic B-cells from a normal individual were fused with the B5-6T heteromyeloma cell line to generate hybridomas. As an assay for general β-pleated sheet binding activity plate-immobilized recombinant λ6-variable domain light chain J to (λ6-LC) was used in fibrillar form, which excluded the possibility of cloning any sequence-directed auto-antibody. The antibody F1 was cloned by limited dilution subcloning. F1 bound to fibrillar λ6-LC and Aβ-fibrils with similar affinity of about 20 nM, but not to non-amyloid elastine aggregates, confirming the desired pan-amyloid specificity (FIG. 3A). Since transgenic animal model data support increasingly a predominant toxic role for low molecular weight Aβ oligomer aggregates over mature fibrils a dityrosine cross-linked Aβ preparation (CAPS) was also used in binding studies as a biochemical proxy for transiently stable Aβ oligomeric protofibrils. F1 also bound CAPS with a high affinity (FIG. 3A). In contrast, binding to plate-immobilized Aβ monomer was weak and non-specific, since it was not competed for by an excess of Aβ monomer in solution (FIG. 3B).

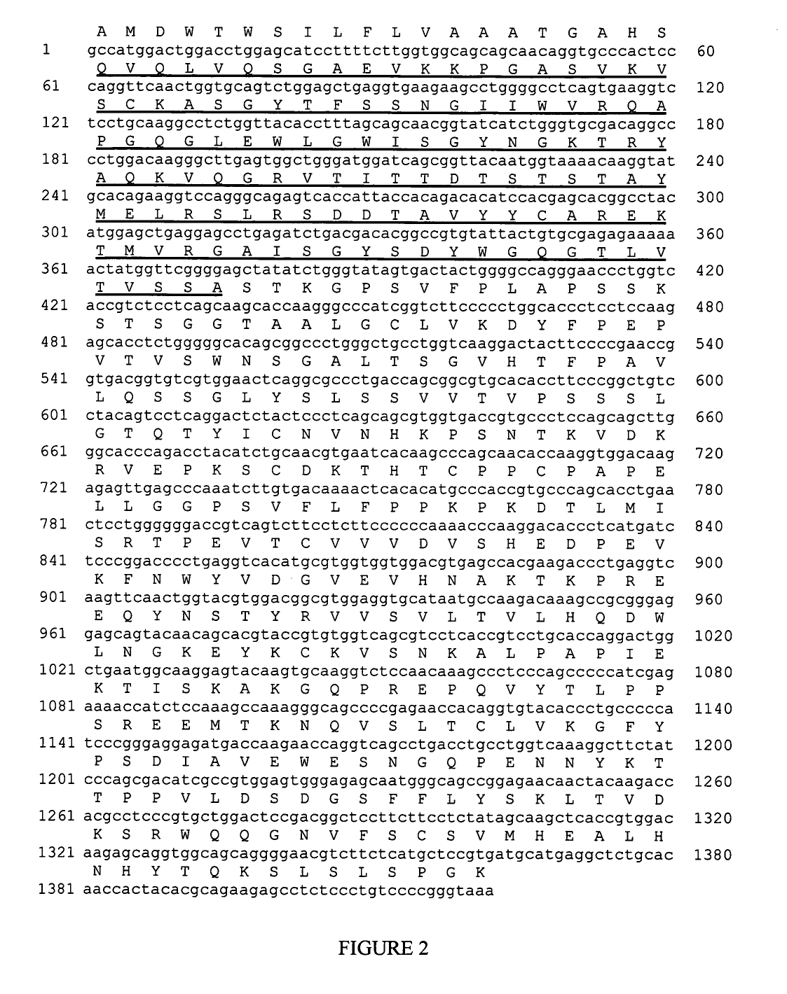

Proteinchemical analysis by gel electrophoresis, by Western-blotting with an anti-light chain pAb, and by ESI-MS revealed surprisingly that the antibody produced by the F1 hybridoma clone consisted only of a heavy chain and lacked a light chain. Detailed cDNA sequence analysis identified F1 as a somatically mutated γ1-heavy chain (IgG1 subclass; FIG. 2) of formula A.

A
GCCATGGACTGGACCTGGAGCATCCTTTTCTTGGTGGCAGCAGCAACA
GGTGCCCACTCCCAGGTTCAACTGGTGCAGTCTGGAGCTGAGGTGAAG
AAGCCTGGGGCCTCAGTGAAGGTCTCCTGCAAGGCCTCTGGTTACACC
TTTAGCAGCAACGGTATCATCTGGGTGCGACAGGCCCCTGGACAAGGG
CTTGAGTGGCTGGGATGGATCAGCGGTTACAATGGTAAAACAAGGTAT
GCACAGAAGGTCCAGGGCAGAGTCACCATTACCACAGACACATCCACG
AGCACGGCCTACATGGAGCTGAGGAGCCTGAGATCTGACGACACGGCC
GTGTATTACTGTGCGAGAGAAAAAACTATGGTTCGGGGAGCTATATCT
GGGTATAGTGACTACTGGGGCCAGGGAACCCTGGTCACCGTCTCCTCA
GCAAGCACCAAGGGCCCATCGGTCTTCCCCCTGGCACCCTCCTCCAAG
AGCACCTCTGGGGGCACAGCGGCCCTGGGCTGCCTGGTCAAGGACTAC
TTCCCCGAACCGGTGACGGTGTCGTGGAACTCAGGCGCCCTGACCAGC
GGCGTGCACACCTTCCCGGCTGTCCTACAGTCCTCAGGACTCTACTCC
CTCAGCAGCGTGGTGACCGTGCCCTCCAGCAGCTTGGGCACCCAGACC
TACATCTGCAACGTGAATCACAAGCCCAGCAACACCAAGGTGGACAAG
AGAGTTGAGCCCAAATCTTGTGACAAAACTCACACATGCCCACCGTGC
CCAGCACCTGAACTCCTGGGGGGACCGTCAGTCTTCCTCTTCCCCCCA
AAACCCAAGGACACCCTCATGATCTCCCGGACCCCTGAGGTCACATGC
GTGGTGGTGGACGTGAGCCACGAAGACCCTGAGGTCAAGTTCAACTGG
TACGTGGACGGCGTGGAGGTGCATAATGCCAAGACAAAGCCGCGGGAG
GAGCAGTACAACAGCACGTACCGTGTGGTCAGCGTCCTCACCGTCCTG
CACCAGGACTGGCTGAATGGCAAGGAGTACAAGTGCAAGGTCTCCAAC
AAAGCCCTCCCAGCCCCCATCGAGAAAACCATCTCCAAAGCCAAAGGG
CAGCCCCGAGAACCACAGGTGTACACCCTGCCCCCATCCCGGGAGGAG
ATGACCAAGAACCAGGTCAGCCTGACCTGCCTGGTCAAAGGCTTCTAT
CCCAGCGACATCGCCGTGGAGTGGGAGAGCAATGGGCAGCCGGAGAAC
AACTACAAGACCACGCCTCCCGTGCTGGACTCCGACGGCTCCTTCTTC
CTCTATAGCAAGCTCACCGTGGACAAGAGCAGGTGGCAGCAGGGGAAC
GTCTTCTCATGCTCCGTGATGCATGAGGCTCTGCACAACCACTACACG
CAGAAGAGCCTCTCCCTGTCCCCGGGTAAA

The corresponding protein sequence of F1 is represented by formula B (single letter code for amino acids):

B
AMDWTWSILFLVAAATGAHSQVQLVQSGAEVKKPGASVKVSCKASGYT
FSSNGIIWVRQAPGQGLEWLGWISGYNGKTRYAQKVQGRVTITTDTST
STAYMELRSLRSDDTAVYYCAREKTMVRGAISGYSDYWGQGTLVTVSS
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
K
RVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
D
HQDWLNGKEYKCKVSNKALPAPIEKTISKAKGQPREPQVYTLPPSREE
L
MTKNQVSLTCLVKGFYPSDIAVEWESNGQPENNYKTTPPVLDSDGSFF
LYSKLTVDKSRWQQGNVFSCSVMHEALHNHYTQKSLSLSPGK

Comparison of the F1 sequence with the known human IgG1 heavy chain sequence revealed that it contained three conservative mutations (underlined amino acids in formula B, amino acids corresponding to the original IgG1 sequence depicted above).

To determine whether the lack of a complementary light chain contributed to the pan-amyloid binding property, isolated heavy chains (HC) from two unrelated monoclonal mAbs directed against botulinum neurotoxin were prepared by standard methods: IgG1 13A (γ1-HC 13A), and IgG2 30B (γ2-HC 30B). Both heavy chain preparations also had high pan-amyloid affinity to the λ6-LC as well as the Aβ fibrils and the CAPS-dimer (Table 2), and similar to F1 (FIG. 3C,D), but unlike the intact parent antibodies, which had no affinity. This establishes that the subclass of the HC is immaterial to the binding activity. Amyloidogenic conformer binding activity of Ig heavy chains was not an artifact of hybridoma expression, since polyclonal HC prepared from immunoglobulin fractions of normal human sera from several different subjects also exhibited an about 60-fold higher affinity to Aβ fibrils than the intact parent IgGs (FIG. 3E).

TABLE 2
Monoclonal human IgG and HC binding to amyloid fibrils and CAPS.

| | | Aβ fibrils | | CAPS | | LC fibrils | | IAPP Fibrils | |
|---|---|---|---|---|---|---|---|---|---|
| | | | Max. | | Max. | | Max. | Max. | |
| | | EC₅₀ | Eu³⁺ | EC₅₀ | Eu³⁺ | EC₅₀ | Eu³⁺ | Eu³⁺ | EC₅₀ |
| Ig | HC | (nM) | (fmoles) | (nM) | (fmoles) | (nM) | (fmoles) | (fmoles) | (nM) |
| HC F1 | γ₁ | 23 ± 0.2 | 249 ± 16 | 28 ± 0.1 | 250 ± 3.0 | 24 ± 0.3 | 195 ± 14 | 40 ± 6.2 | 110 ± 11 |
| IgG F2 | γ₁ | 145 ± 1.0 | 84 ± 6.0 | 330 ± 3.0 | 58 ± 7.0 | 21 ± 0.3 | 228 ± 16 | >1000 | >10 |
| IgG 13A | γ₁ | >1000 | >30 | >1000 | >10 | >1000 | >20 | >1000 | >15 |
| HC 13A | γ₁ | 14 ± 0.01 | 261 ± 2.0 | n.d. | n.d. | 65 ± 0.8 | 306 ± 20 | n.d. | n.d. |
| IgG 30B | γ₂ | >1000 | >40 | n.d. | n.d. | >1000 | >50 | n.d. | n.d. |
| HC 30B | γ₂ | 109 ± 0.3 | 430 ± 7 | n.d. | n.d. | 23 ± 0.2 | 23 ± 0.2 | n.d. | n.d. |

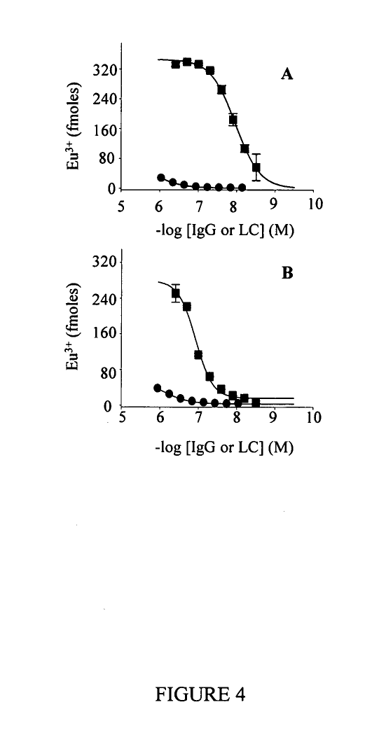

The affinity of isolated heavy chain fragments of antibodies to generic conformations displayed by all fibrillar proteins/peptides relative to the intact antibody is not limited to heavy chains, but is shared by all isolated chains, heavy or light chains. A similar preferential affinity over intact parent antibodies for plate-bound fibrillar Aβ peptide was demonstrated with isolated light chains (LC) of the non-Aβ mAbs 13A (FIG. 4A) and 30B (FIG. 4B), whereby the LC 13A was about an order of magnitude more potent. This demonstrates moderate differences in binding affinity of LCs, since the LC 13A is of the λ3 subclass while the LC 30B is of the λ1 subclass.

The binding of the γ1-HC antibody F1 to fibril Aβ is effective to prevent the propagation of Aβ fibril growth with a half-maximal effect near 1 μM, while the heavy chain of mAb 13A is less potent (FIG. 4A). This shows that there are differences in the functional efficacy of isolated heavy chains, even within the same subclass, whereby F1 is preferred. As expected, the intact mAb 13A is as ineffective as the inert control bovine serum albumin. Such differences in activity are also evident for isolated light chains.

The biological significance of the inactivation of β-pleated sheet conformers is exemplified in the reversal of the inhibition by Aβ peptide of the process of longterm potentiation (LTP) in the rat hippocampus in vivo, generally regarded as a correlate to learning and memory circuitry. Intracerebroventricular application of Aβ peptide essentially abrogated LTP as a measure of the strength of synaptic transmission (FIG. 4B), whereas the co-administration of the isolated heavy chain F1, but not of the intact control mAb 13A, significantly antagonized the suppression of LTP by Aβ (FIG. 4C).

In one aspect of the invention isolated LC or HC, preferably F1, are used to modify pathological processes in animal models based on amyloid formations. Various demonstrations of the efficacy of heavy chain antibodies, in particular F1, to inhibit the deleterious effects of fibrillar disease proteins and peptides in established models are easily conceived by those skilled in the art. Without limiting the scope of the disclosure, various transgenic mouse models well established in the art, such as CNS disease models presenting with cerebral Aβ amyloidosis and/or tau pathology for AD, synucleopathy for Lewy body variants of dementia and Parkinson's disease (PD), or prion disease (various forms of spongiform encephalitis, scrapie, etc.) can be treated with isolated Ig light or heavy chains, including but not limited to F1. In such treatments the isolated HC/LC is administered i.v. in doses ranging from 0.01 to 100 mg/kg, e.g. by injection into the femoral artery. Dosing may be repeated in weekly or monthly intervals as required by the particular model. The efficacy of the treatment can be asserted by histochemical analysis of brain tissues with various pathological markers known in the art, e.g typical stains for amyloid (Congo Red, Thioflavin T), silver stains for aggregated proteins, or specific antibodies for epitopes associated with the respective pathology. Reduction of the respective pathology is expressed in reference to vehicle-treated control animals. Functional read-outs can be learning and memory tests (e.g. Morris water maze, novel object recognition, fear conditioning) in cortical pathologies for AD models, or motor skill tests for PD models (rotarod test, beam balance test, grip strength test) as are well-established in the art.

In another aspect of the invention the amyloidosis in an animal model can be peripheral, such as any of the amyloidoses listed in Table 1. The antibody fragments of the invention are applied i.v. in doses ranging from 0.01 to 100 mg/kg. Efficacy is verified by measuring the extent of inhibition of the amyloidosis in the respective target organ relative to vehicle treated control animals, and improvement of functional parameters, such as survival.

In another aspect of the invention human subjects suffering from an amyloidosis are treated with an isolated LC or HC, preferably F1. The HC or LC can be monoclonal, as derived from a hybridoma cell line or produced by means of recombinant cDNA expression, or can be polyclonal, as obtained from purified immunoglobulin preparations from blood. HC can be derived from the IgGA, IgD, IgE, IgG, or IgM subtype. LC can be of the lambda (λ) or the kappa (κ) subtype. The source of the antibody is preferably human. Mixtures of isolated chains can also be entertained. The treatment can be therapeutic after diagnosis of clinical symptoms of the respective amyloid disease, or prophylactic after predictive diagnosis based on suitable genetic tests (e.g. APP or PS1,2 mutations for familial AD, Tau mutations in various tauopathies; LRRK2, parkin, PINK1, or synuclein mutations for PD), blood tests, CSF marker profile for CNS diseases (e.g. tau ELISA), familial history, or imaging methods (e.g. brain Aβ amyloidosis with Pittsburgh compound B [Klunk et al., Ann. Neurol. 55, 306-319 (2004)]). The isolated antibody chain can be applied as a monomer or a homodimer, or as a mixture of both.

In another aspect of the invention the isolated antibody chain can also be modified by specific mutations designed to reduce disulfide bridging in order to prevent the isolated antibody chain from homo/hetero-dimerization, preferably by exchanging one or several of the Cysteine residues involved in intermolecular chain crosslinking in a HC or a LC (e.g. Cys227, Cys233, and Cys236 in F1) by a conservative exchange against Alanine or Serine, using mutagenesis of the cDNA with any the methods known by those skilled in the art, and recombinant expression of the mutant protein.

In another aspect of the invention an active fragment of a HC or LC can be applied to treat a disease caused by an amyloidosis, since the activity of isolated chains is evidently independent of subclass and the precise nature of the variable domains that are critical for recognition of sequence-directed epitopes. It is thus conceived that the absence of pairing with a complementary chain is solely sufficient for activity. Preferably such a fragment consists of any of the C_{H}, C_{L}, V_{H} or V_{L} subdomains, or combinations thereof, as can be conveniently prepared by truncation of the cDNA of the single antibody chain and recombinant expression of the corresponding protein product. Alternatively, such fragments can be chosen as can be conveniently obtained by limited protease digestion of the full length chain, e.g. in the hinge region between HC subdomains C_{H}1 and C_{H}2 by trypsin or papain, using conditions well known in the art. One skilled in the art would understand that any portion of the heavy chain or the light chain may also be sufficient to obtain the desired binding to amyloid. Accordingly, one of skill in the art would be motivated to remove portions of the heavy chain, from either or both the amino or carboxy terminal ends, to determine what portions of the heavy chain may be removed while still maintaining efficacy. Similarly, one of skill in the art would be motivated to remove portions of the light chain, from either or both the amino or carboxy terminal ends, to determine what portions of the light chain may be removed while still maintaining efficacy. It is also apparent to anyone skilled in the art that fragments and/or subdomains of isolated HC and LC can be recombined into hybrid single chain antibodies by fusing the respective cDNA sequences and expression of the recombinant protein in a suitable host.

The isolated antibody chains of the invention can be applied intramuscularly, subcutaneously, or intravenously by means of a bolus injection or by infusion at controlled flow rates. For all of these forms of application the antibody will be in the form of an aqueous solution. Such solutions can be reconstituted from a sterile lyophilized form of the antibody chain, from a concentrate, or can already be in the form of use in defined dosing packages. Vehicles may contain physiological isotonic salt and buffer constituents (e.g. Ringer's solution), wetting agents, as are commonly known in the art, and may further contain non-aqueous solvents designed by the FDA as “Generally Accepted As Safe” (GRAS) vehicles to enhance solubility and/or stability. Vehicles may further contain preservatives to prevent bacterial contamination acceptable for human use.

The isolated antibody chains of the invention can be applied in doses ranging from 0.01 to 100 mg/kg. Doses may be adjusted to individual needs to minimize side effects according to the judgment of the skilled physician. The frequency of dosing may range from two applications per month up to one application every three months, in accordance with schedules common to established antibody treatments. The most appropriate dosing regimen will be determined by the physician by monitoring acute clinical signs of the disease and their progression over time, or by surrogate markers effects in bodily fluids, or by imaging methods. In the chronic neurodegenerative diseases causing dementia (e.g. AD) a regular schedule of standardized neuropsychometric test batteries (MMSE scores, ADAS-Cog scale) will be used to determine the most effective dosing regimen for nay given patient. As an adjuvant criterion, the reversal of disease associated changes of biological markers in CSF of patients (e.g. Aβ, tau, and phospho-tau levels in AD) can be monitored. For movement disorders, like PD, the standard unified rating scale may be employed to identify the dosing regimen where the progression of the disease is minimized.

In one aspect of the invention treatment with the isolated antibody chains is performed in combination with established standard therapies. For the neurodegenerative diseases the antibody chains of the invention may be administered in conjunction with one or several of the following agents: Acetylcholine esterase inhibitors, nicotinic agonists, Memantine, APP β- and γ-secretase inhibitors, Aβ modulators, sequence-directed Aβ antibodies (passive immunization), kinase inhibitors, L-DOPA, MAO inhibitors, dopamine receptor agonists, dopamine reuptake inhibitors.

Isolated chain antibodies can be prepared in several ways. Commercially available purified Ig preparations consisting essentially of polyclonal intact antibodies can be subjected to treatment with a reducing agent, such as dithiothreitol (DTT), to break up disulfide bridges, followed by size exclusion chromatography under acidic conditions [McLaughlin and Solomon, J. Immunol. 113, 1369-1372 (1974)]. Mild alkylation, e.g. carboxymethylation can be employed to facilitate dissociation of antibody chains from the intact antibody assembly, which does not affect the amyloid-binding properties of isolated chains. This method can also be employed with monoclonal antibodies obtained from hybridomas. Another method is the expression of the isolated cDNA of an antibody LC or HC, preferably F1, in a suitable vector and host organism known to those skilled in the art. Expression can be performed in hybridomas, mammalian cell culture, e.g. CHO cells, insect baculovirus systems, or in plants, e.g. tobacco using the tobacco mosaic virus (TMV) as a vector. The purification of the expressed isolated chain is then accomplished by various chromatographic means known in the art, preferably affinity chromatography for high purity.

The invention is further described in the following non-limiting examples.

### Examples

### Example 1

### Preparation of Isolated Heavy Chain Antibody F1 from a Hybridoma Cell Line

The F1 secreting hybridoma cell line, generated from fusion of a splenic mononuclear cell with the B5-6T heteromyeloma cell line, was plated at a density of 5×105 cells/ml in 100 ml serum-free culture medium (IS MAB-CD, Irvine Scientific), and incubated for 5 days in a 500 ml roller bottle at 37° C. Supernatants were filtered over a sterile 0.22 μm filter and purified on a protein G-Sepharose column. The fraction eluted with a 100 mM glycine.HCl buffer at pH 2.7 contained the 55 kD heavy chain F1 at about 90% purity per SDS-PAGE.

### Example 2

### Separation of Heavy and Light Chains of Immunoglobulins

A commercial IgG fraction is treated with 75 mM DTT in 6M guanidinium hydrochloride at pH 8 and 0.1M iodoacetamide for 1 hr at 37° C. Separation of the chains was accomplished by size exclusion chromatography on a 2.5×100 cm Sephadex G100 Superfine column equilibrated in 10% acetic acid. Product fractions were pooled according to SDS-PAGE analysis of eluate fractions for light and heavy chains, respectively. Fractions thereafter were renatured by dialysis into 10 mM phosphate-buffered saline (PBS) with 0.02% sodium azide.

A similar process can be applied to obtain the isolated heavy chain F1 from hybridoma supernatants in monomeric form.

### Example 3

### Binding of Antibody Heavy Chain F1 to Fibrillar Aβ1-40

High binding activated microtiter plate wells were coated with 400 ng of fibrillar Aβ1-40 peptide, which was prepared by aggregation in 10 mM PBS of Aβ1-40 peptide, previously disaggregated by the trifluoro acetic acid/hexafluoroisopropanol procedure [Adekar et al., J. Biol. Chem. 285, 1066-1074 (2010)]. After blocking with 1% bovine serum albumin (BSA), F1 was serially diluted in 10 mM PBS/1% BSA and applied in 100 μl aliquots to coated wells. After incubation for 1 hr, wells were washed, and incubated with biotinylated goat anti-human IgG as a secondary antibody for 1 hr. Thereafter bound HC F1 was determined with a Eu³⁺-streptavidine conjugate followed by the release enhancement reagent (EuLISA) and measuring the amount of Eu³⁺ by time-resolved fluorescence (Victor 1420 multilabel counter) against a standard curve established with known concentrations of Eu³⁺.

Microtiter wells can be coated with other amyloid proteins aggregates for similar binding experiments [e.g. J to light chain, cross-linked Aβ peptide dimers: Adekar et al., J. Biol. Chem. 285, 1066-1074 (2010)], and other isolated Ig light or heavy chains can be used for binding with the same procedure.

### Example 4

### Inhibition of Aβ Peptide Fibril Extension by Heavy Chain F1

High binding microtiter plate wells were coated with 400 ng sonicated Aβ1-40 fibrils, and serially diluted antibody heavy chain F1 was applied in concentrations from 10 nM to 1 μM in 10 mM PBS. Thereafter 50 nM of soluble biotinylated Aβ peptide monomers were added. After 3 hrs of incubation wells were washed, and fibril-recruited biotinylated Aβ peptide was quantified in a EuLISA using Eu³⁺-streptavidine conjugate and time-resolved fluorescence detection (Victor 1420 multilabel counter).

### Example 5

### Neutralization of the Suppression of LTP by Aβ1-42 Peptide with the Antibody Heavy Chain F1 In Vivo

Using urethane-anesthetized adult male Wistar rats, single pathway recordings of field postsynaptic potentials (EPSPs) from the stratum radiatum in the CA1 area of the hippocampus were determined in response to stimulation of the ipsilateral Schaffer collateral-commissural pathway according to an established protocol [Klyubin et al., Eur. J. Neurosci. 19, 2839-2846 (2004)]. Test EPSPs were evoked at a frequency of 0.033 Hz and at a stimulation intensity adjusted to give an EPSP amplitude of 50% of maximum. The high frequency stimulation protocol for inducing LTP consisted of 10 trains of 20 stimuli, an interstimulus interval of 5 ms (200 Hz), and an intertrain interval of 2 s. The intensity was increased to give an EPSP of 75% of the maximum amplitude during high frequency stimulation. To inject samples, a cannula was implanted in the lateral cerebral ventricle (coordinates: 1 mm lateral to the midline and 4 mm below the surface of the dura) just before electrode implantation. Soluble synthetic Aβ1-42 was prepared as described [Klyubin et al., Eur. J. Neurosci. 19, 2839-2846 (2004)]. Briefly, a 5-μl aliquot of 40 pmole of peptide with or without ˜3-6 μg of HC F1 was intracerebroventricularly injected 10 min before high frequency stimulation over a 2-min period. Control vehicle injections comprised sterile water. LTP was expressed as the mean±S.E. percentage base-line field EPSP amplitude recorded over at least a 30-min base-line period. Similar results were obtained when the EPSP slope was measured. The statistical effect of the HC F1 was evaluated using paired and unpaired Student's t-tests.

While preferred embodiments of the invention have been described in detail, it will be apparent to those skilled in the art that the disclosed embodiments may be modified. It is intended that such modifications be encompassed in the invention. Therefore, the foregoing description is to be considered to be exemplary rather than limiting, and the scope of the invention is that defined by the following claims.

1. A method for selectively binding aggregated amyloidogenic proteins comprising exposing the aggregated amyloidogenic proteins to an antibody chain that is capable of binding to aggregated amyloidogenic proteins, wherein the antibody chain comprises a heavy chain of an immunoglobulin, a light chain of an immunoglobulin, or a portion of a heavy or light chain of an immunoglobulin, and wherein, if the antibody chain comprises a portion of or a complete immunoglobulin heavy chain, the antibody chain does not contain an immunoglobulin light chain and, if the antibody chain comprises a portion of or a complete immunoglobulin light chain, the antibody chain does not contain an immunoglobulin heavy chain, and permitting the antibody chain to bind to the aggregated amyloidogenic proteins.

2. The method of claim 1 wherein the antibody chain comprises a complete heavy chain or light chain of an immunoglobulin.

3. The method of claim 2 wherein the antibody chain has an amino acid sequence as shown in FIG. 2.

4. The method of claim 2 wherein the antibody chain has an amino acid sequence as shown in FIG. 2 wherein one or more of the cysteine residues at amino acid positions 227, 233, and 236 have been replaced by an alanine or serine residue.

5. The method of claim 1 wherein the antibody chain comprises a portion of a heavy or light chain of an immunoglobulin.

6. The method of claim 5 wherein the antibody chain has an amino acid sequence that is a portion of the amino acid sequence shown in FIG. 2.

7. The method of claim 6 wherein the antibody chain has an amino acid sequence that is a portion of the amino acid sequence shown in FIG. 2 wherein one or more of the cysteine residues at amino acid positions 227, 233, and 236 have been replaced by an alanine or serine residue.

8. The method of claim 5 wherein the antibody chain comprises one or more of the C_{H}1, C_{H}2, C_{H}3, or V_{H} domains of the heavy chain.

9. The method of claim 5 wherein the antibody chain comprises one or more the C_{L} or V_{L} domains of the light chain.

10. The method of claim 1 wherein the aggregated amyloidogenic proteins are selected from the group consisting of Aβ peptides and tau protein.

11. An isolated antibody chain comprising a portion or all of the amino acid sequence of FIG. 2, wherein the antibody chain does not contain an immunoglobulin light chain, and wherein the antibody chain selectively binds to aggregated amyloidogenic proteins.

12. The isolated antibody of claim 11 that comprises the V_{H} variable domain as shown in FIG. 2.

13. The isolated antibody of claim 11, wherein one or more of the cysteine residues at positions 227, 233, and 236 of FIG. 2 have been replaced by an alanine or a serine residue.

14. The isolated antibody of claim 11 that comprises amino acids 1 to 300 of the amino acid sequence of FIG. 2.

15. A method for ameliorating the signs or symptoms of an amyloid related disorder in an individual suffering from such a disorder comprising administering to the individual an antibody chain that is capable of binding to aggregated amyloidogenic proteins, wherein the antibody chain comprises a heavy chain of an immunoglobulin, a light chain of an immunoglobulin, or a portion of a heavy or light chain of an immunoglobulin, and wherein, if the antibody chain comprises a portion of or a complete immunoglobulin heavy chain, the antibody chain does not contain an immunoglobulin light chain and, if the antibody chain comprises a portion of or a complete immunoglobulin light chain, the antibody chain does not contain an immunoglobulin heavy chain, in an amount sufficient to ameliorate the signs or symptoms of the disorder.

16. The method of claim 15 wherein the antibody chain has a portion or all of the amino acid sequence shown in FIG. 2.

17. The method of claim 16 wherein one or more of the cysteine residues at positions 227, 233, and 236 of FIG. 2 have been replaced by an alanine or a serine residue.

18. The method of claim 16 wherein the antibody chain comprises the V_{H} variable domain as shown in FIG. 2.

19. The method of claim 16 wherein the antibody chain comprises the amino acid sequence shown in FIG. 2.

20. The method of claim 15 wherein the disorder is Alzheimer's disease.
